(19) United States
(12) Patent Application Publication
(10) Pub. No.: US 2003/0069564 A1
(43) Pub. Date: Apr. 10, 2003

(54) Guard for contoured medical instruments

(76) Inventors: David M. Webb, Blaine, MN (US); Frederick E. Berg, Hopkins, MN (US)

Correspondence Address: Theodore M. Magee, WESTMAN CHAMPLIN & KELLY, International Centre - Suite 1600, 900 South Second Avenue, Minneapolis, MN, 55402-3319, US

(21) Appl. No.: 09/971,191

(22) Filed: Oct. 4, 2001

(51) Int. Cl. A61B 17/00

(52) U.S. Cl. 606/1

(57) ABSTRACT

A guard for medical instruments includes an opening defining portion that defines an opening capable of receiving a portion of a medical instrument. The guard also includes a base portion that maintains the guard in a stable position such that the opening defining portion is separated from a surface that the guard is resting on. In other embodiments, the guard includes an opening that is large enough to permit the insertion of the end of a medical instrument and a contoured portion that at least partially follows the contour of the end of the medical instrument.

### FIELD OF THE INVENTION

The present invention is related to medical instruments. In particular, the present invention is related to guards for protecting medical instruments from damage.

### BACKGROUND OF THE INVENTION

Medical instruments such as scalpels, forceps, clamps and retractors are sometimes subject to rough handling that can cause precisely formed ends of the instruments to chip.

To prevent such damage, the art has developed sleeves of formed plastic that slip over the ends of many instruments. Although these sleeves work well for instruments that are not contoured such as a scalpel or a simple clamp, they are less than ideal for contoured instruments such as retractors, which typically have hooked or L-shaped ends. In particular, users find it difficult or impossible to insert these contoured instruments into the prior art sleeves.

The art has developed one guard for protecting retractors. This guard is formed by cutting a hole in the side wall of a piece of plastic tubing. The retractor is then inserted in the sidewall hole. Although such a guard provides sufficient space for the retractor, it is not ideal because the guard tends to roll away from the user making it difficult to control.

### SUMMARY OF THE INVENTION

A guard for medical instruments includes an opening defining portion that defines an opening capable of receiving a portion of a medical instrument. The guard also includes a base portion that maintains the guard in a stable position such that the opening defining portion is separated from a surface that the guard is resting on.

In other embodiments, the guard includes an opening that is large enough to permit the insertion of the end of a medical instrument and a contoured portion that at least partially follows the contour of the end of the medical instrument.

### BRIEF DESCRIPTION OF THE DRAWINGS

FIG. 1 is a perspective view of one type of retractor that can be protected by a guard of the present invention.

FIGS. 2, 3, 4, 5, 6 and 7 provide front, side, top, bottom, top perspective and bottom perspective views, respectively, of one embodiment of a guard of the present invention.

FIG. 8 provides a partial cut-away perspective view of the guard of FIG. 2 with a retractor inserted in the guard.

FIGS. 9, 10, 11, 12, 13, and 14 provide front, side, top, bottom, top perspective and bottom perspective views, respectively, of a second embodiment of a guard of the present invention.

FIGS. 15, 16, 17, 18, 19, and 20 provide front, side, top, bottom, top perspective and bottom perspective views, respectively, of a third embodiment of a guard of the present invention.

FIGS. 21, 22, 23 and 24 provide front, side, top, and top perspective views, respectively, of a fourth embodiment of a guard of the present invention.

FIG. 25 provides a partial cut-away side view of the guard of FIG. 21 with a retractor inserted in the guard.

FIGS. 26 and 27 provide side and top perspective views, respectively, of a fifth embodiment of a guard of the present invention.

FIGS. 28 and 29 provide front and bottom perspective views, respectively, of a sixth embodiment of a guard of the present invention.

FIGS. 30, 31, 32, 33, and 34 provide front, side, front cross-sectional, top, and bottom views, respectively, of a seventh embodiment of a guard of the present invention.

FIGS. 35, 36, 37, and 38 provide top, side, front, and top perspective views, respectively, of an eighth embodiment of a guard of the present invention.

FIG. 39 provides a partial cut-away side view of the guard of FIG. 35 with a retractor inserted in the guard.

### DETAILED DESCRIPTION OF ILLUSTRATIVE EMBODIMENTS

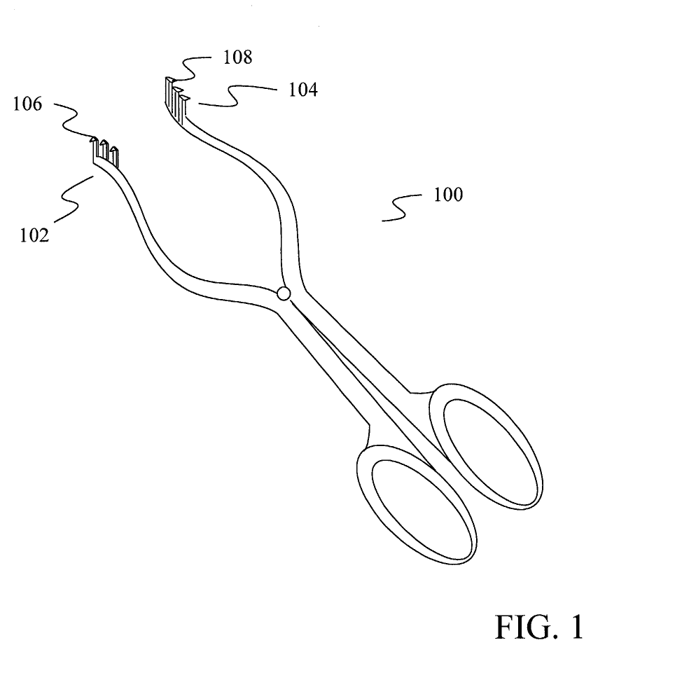

The present invention is a guard for a contoured medical instrument such as retractor 100 of FIG. 1. Retractor 100 includes two ends 102 and 104 that include contoured tines such as tines 106 and 108. The guards of the present invention are designed to protect contoured ends such as ends 102 and 104 from physical damage due to rough handling.

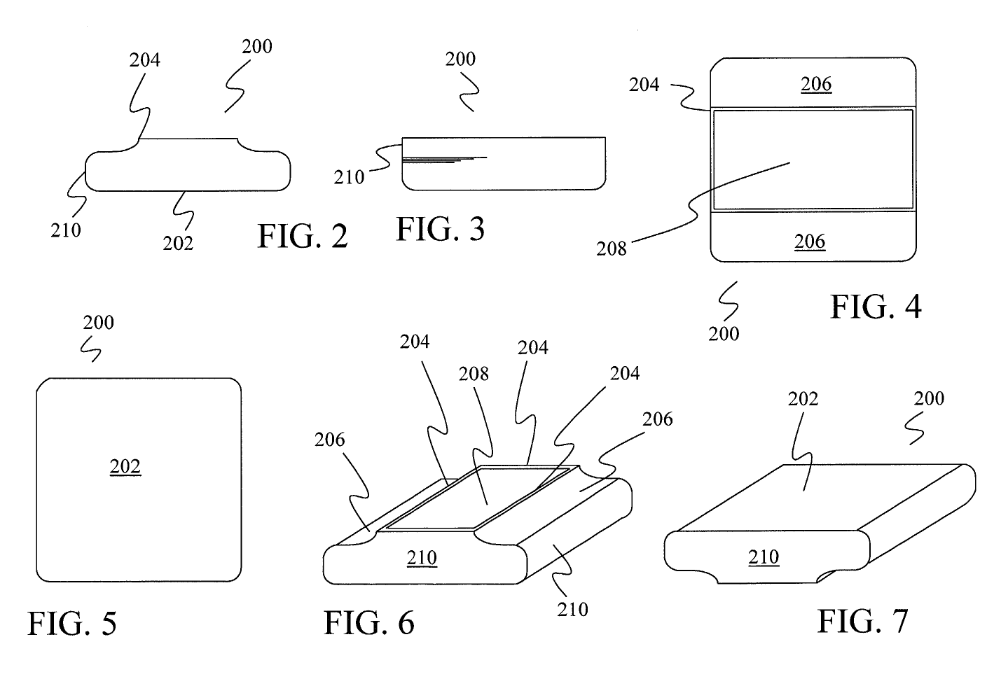

FIGS. 2, 3, 4, 5, 6 and 7 provide front, side, top, bottom, top perspective and bottom perspective views, respectively, of a guard 200 of one embodiment of the present invention.

Guard 200 includes a base portion 202 and an opening defining portion 204. Opening defining portion 204 is part of a top portion 206 and defines an opening 208 that accepts the ends of a medical instruments such as retractor 100 of FIG. 1. A side section 210 extends between base portion 202 and top portion 206. In guard 200, base portion 202 has a flat planar surface and top portion 206 has a curved surface.

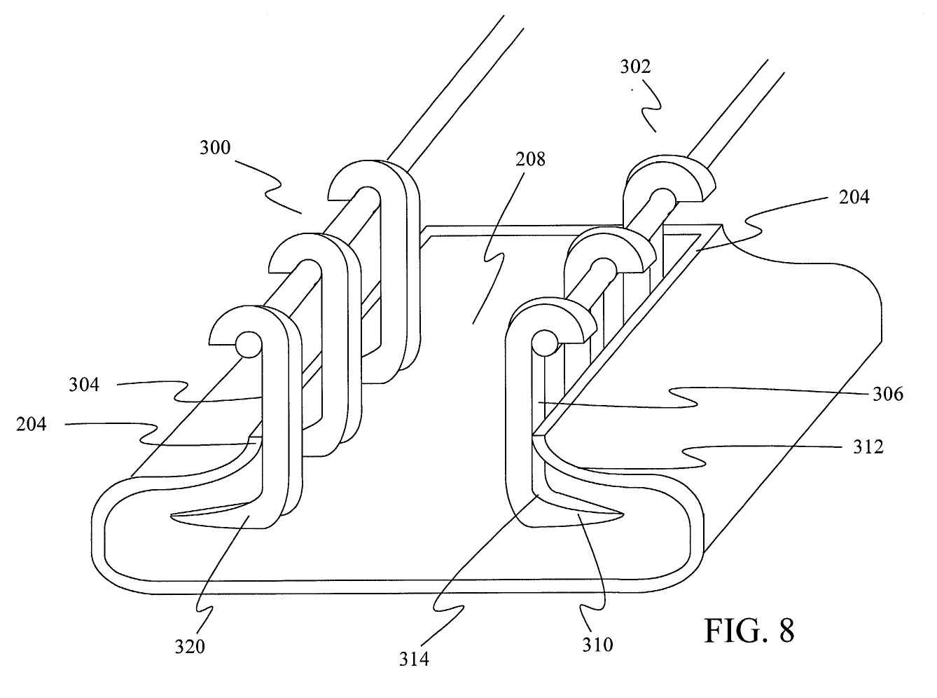

In one embodiment, base 202 is 28 mm by 28 mm (1.06 inches by 1.06 inches) and the vertical distance from opening defining portion 204 to base 202 is 9 mm (0.36 inches). The opening in this embodiment is 25 mm by 13 mm (1 inch by 0.52 inch). The guard in this embodiment has a generally uniform wall thickness of approximately 0.8 mm (0.032 inches) FIG. 8 is a partially cut-away perspective view of guard 200 with ends 300 and 302 of a retractor inserted in the guard. To insert ends 300 and 302, the retractor ends are collapsed together so that they can both fit in opening 208. Once the tines are past opening defining area 204, the retractor is expanded so that sides of the ends of the retractor such as sides 304 and 306 of times 310 and 320 engage opening defining area 204.

As can be seen in FIG. 8, top portion 206 provides a contoured portion that at least partially follows the contour of ends 300 and 302. In particular, contour 312 of top portion at least partially follows the contour formed by the bend in the tines of ends 300 and 302 such as bend 314 of tine 310.

When ends 300 and 302 are inserted in guard 200, they are protected from striking hard surfaces directly and thus are less likely to be damaged. Because the guard is contoured and has a sufficiently large opening, it is relatively easy to insert the ends of the retractor. The contouring of the guard also helps to keep the ends of the retractor within the guard because the tines engage the top portion of the guard if the retractor is moved relative to the guard.

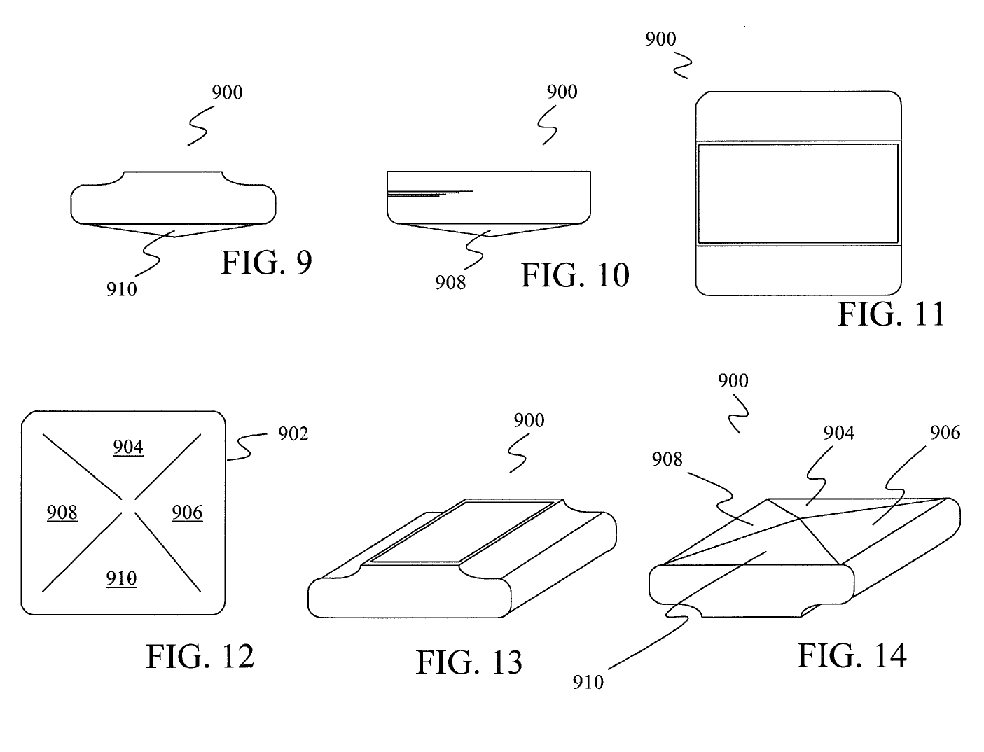

FIGS. 9, 10, 11, 12, 13, and 14 provide front, side, top, bottom, top perspective and bottom perspective views, respectively, of a guard 900 of a second embodiment of the present invention. Guard 900 is similar to guard 200 except that base 902 of guard 900 is formed of four planar surfaces 904, 906, 908, and 910 that form a four-sided pyramid structure that extends below guard 900. Each of the four planar surfaces is capable of maintaining the guard in a stable position when the guard is resting on one of the surfaces. This helps to keep the guard from moving when a user is inserting a medical instrument into the guard and in many causes allows the user to insert the instrument while only using one hand.

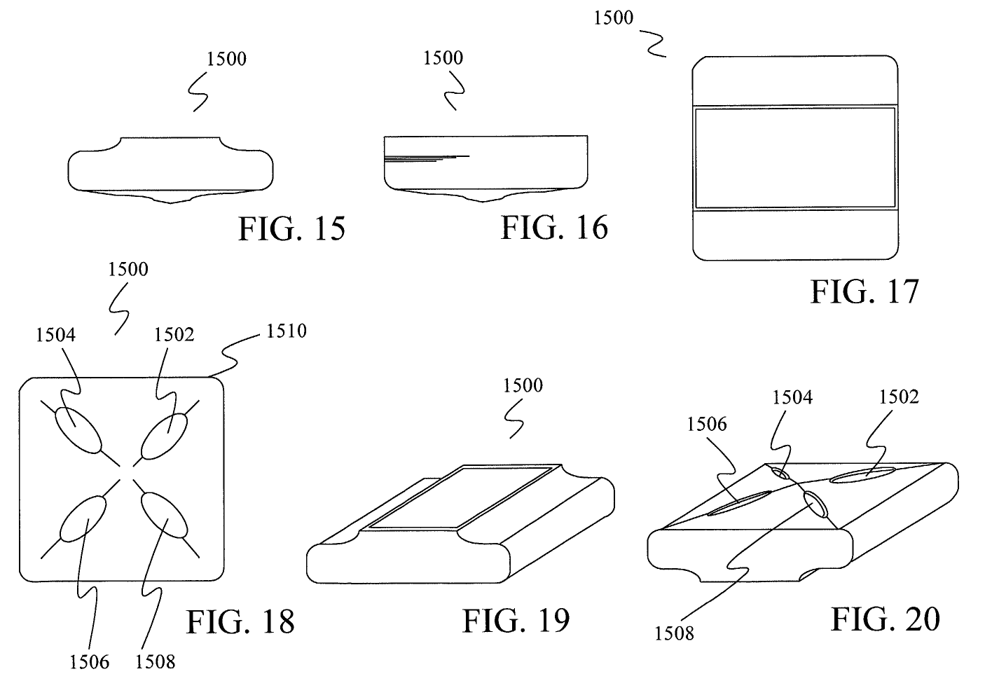

FIGS. 15, 16, 17, 18, 19, and 20 provide front, side, top, bottom, top perspective and bottom perspective views, respectively, of a guard 1500 of a third embodiment of the present invention. Guard 1500 is similar to guard 900 with the addition of four holes 1502, 1504, 1506, and 1508 in base 1510. Holes 1502, 1504, 1506, and 1508 enhance the flow of sterilizing fluids through the guard when the guard and retractor are placed in a sterilization unit. In other embodiments, holes 1502, 1504, 1506 and 1508 are replaced by a single hole at the point where the four sides of the pyramid bottom come together.

Note that guard 1500 is not the only guard designed to be used in a sterilization unit under the present invention and that all of the guards of the present invention may be made of a material designed to withstand sterilization.

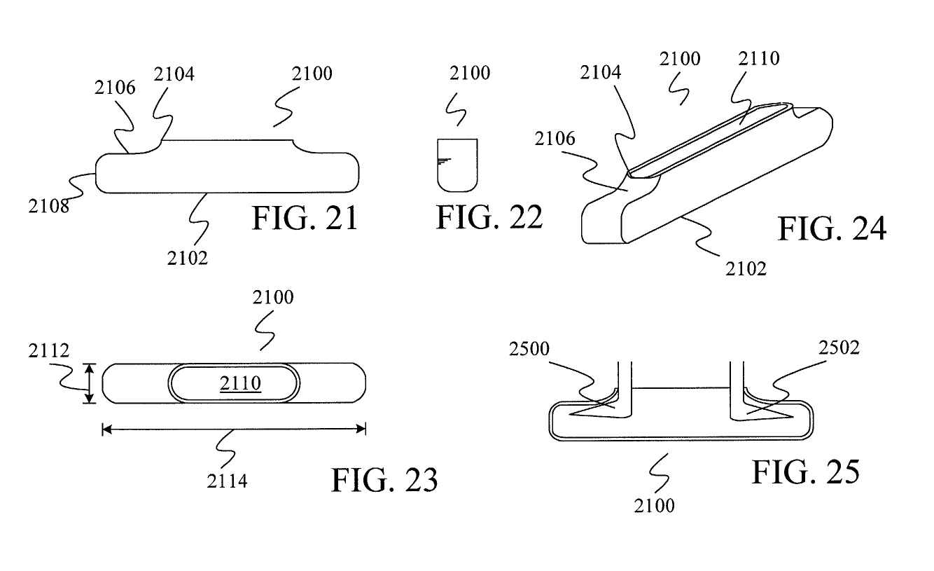

FIGS. 21, 22, 23 and 24 provide front, side, top, and top perspective views, respectively, of a guard 2100 of a fourth embodiment of the present invention. Guard 2100 has a base 2102 and an opening defining region 2104 that forms part of a top section 2106. Opening defining region 2104 defines an opening 2110 into which a medical instrument may be inserted. A side section 2108 extends between base 2102 and top section 2106.

Base 2102 of guard 2100 is planar thereby maintaining the guard in a stable position when the guard is placed on a surface. In other embodiments, base 2102 is formed of two planes that extend downward from side section 2108 and meet at the center of the base along a ridge that extends along the long dimension (length 2114) of the guard.

As can be seen in FIG. 23, guard 2100 has a narrower width 2112 than length 2114. In one embodiment, the width is 12 mm (0.48 inches) and the length is 48 mm (1.92 inches). This makes the well suited for retractors that have a single tine on the end. In one embodiment, guard 2100 has a height of 10 mm (0.4 inches) from base 2102 to opening defining region 2104.

FIG. 25 shows a partial cut-away side view of guard 2100 with two ends 2500 and 2502 of a retractor inserted in the guard. To insert the ends, the retractor is collapsed to bring ends 2500 and 2502 closer together so that they both fit through opening 2110. Once the ends are within the guard, the retractor is expanded to bring the ends into contact with opening defining portion 2104.

Top section 2106 provides a contour that at least partially follows the contour of ends 2500 and 2502 as can be seen in FIG. 25. This helps to keep the guard in place and makes it easier to insert the retractor into the guard.

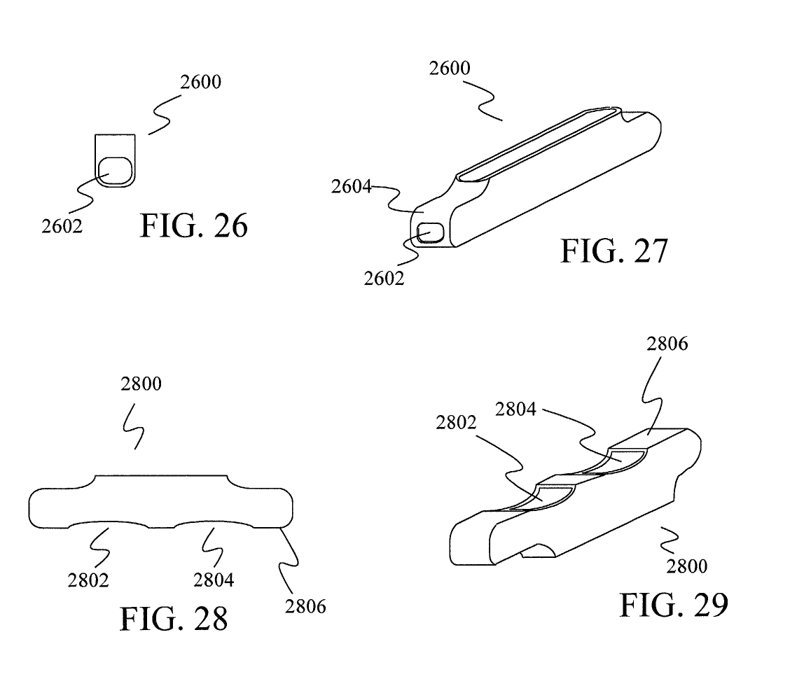

FIGS. 26 and 27 provide side and top perspective views, respectively, of a guard 2600 of a fifth embodiment of the present invention. Guard 2600 is similar to guard 2100 with the addition of side holes such as side hole 2602 located in a side section 2604 of the guard. Side hole 2602 aids the transport of sterilizing fluids through the guard.

FIGS. 28 and 29 provide front and bottom perspective views, respectively, of a guard 2800 of a sixth embodiment of the present invention. Guard 2800 is similar to guard 2100 of FIG. 21 with the addition of two bottom holes 2802 and 2804 formed in the base 2806. In other embodiments, holes 2802 and 2804 are replaced by a single hole in the bottom of the guard.

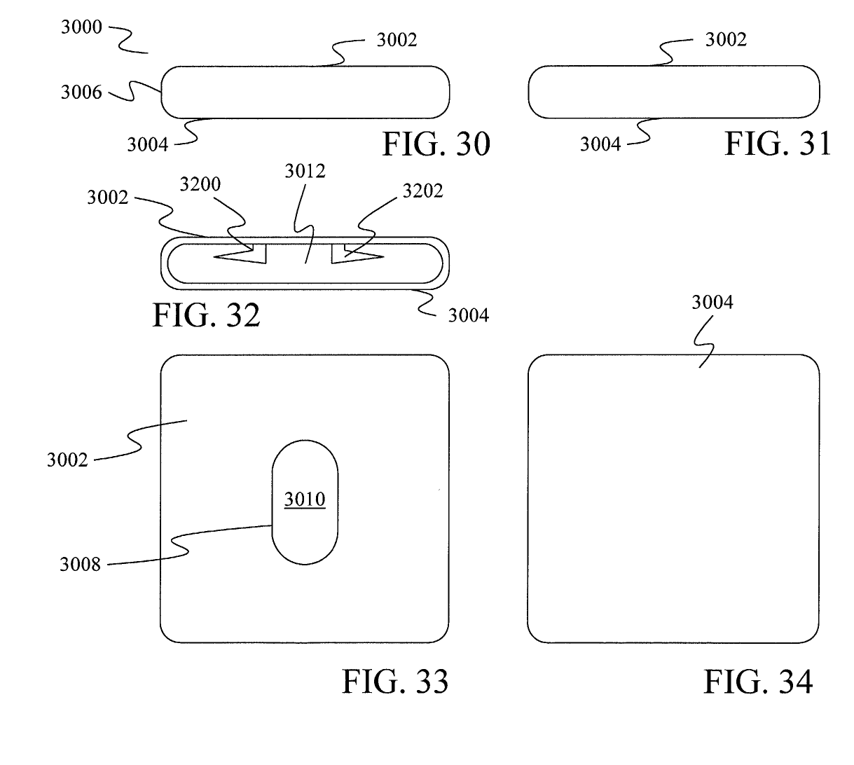

FIGS. 30, 31, 32, 33, and 34 provide front, side, front cross-sectional, top, and bottom views, respectively, of a guard 3000 of a seventh embodiment of the present invention. Guard 3000 includes a planar base 3004 and a planar top section 3002 that are parallel to each other. A side section 3006 extends between base 3004 and top 3002.

As shown in FIG. 32, guard 3000 includes a space 3012 defined between top 3002 and base 3004 into which ends 3200 and 3202 of a retractor may be inserted. In particular, ends 3200 and 3202 are placed in space 3012 by inserting the ends through an opening 3010 defined by an opening defining portion 3008 of top 3002.

In one embodiment, top portion 3002 and bottom portion 3004 are 40 mm by 40 mm (1.6 inches by 1.6 inches) and space 3012 is 3 mm (0.12 inches) measured from base 3004 to top 3002. Opening 3010 in this embodiment is 9 mm by 22 mm (0.36 inches by 0.88 inches).

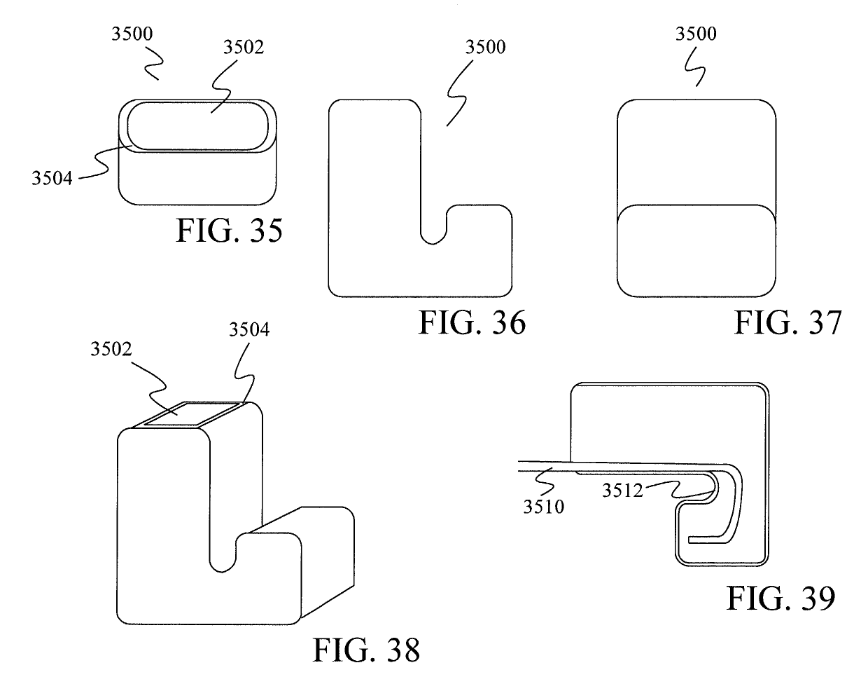

FIGS. 35, 36, 37, and 38 provide top, side, front, and top perspective views, respectively, of a guard 3500 of an eighth embodiment of the present invention. Guard 3500 is designed to protect a retractor that has a single retracting end instead of two opposed retracting ends.

Guard 3500 has an opening 3502 that is defined by an opening defining portion 3504. As shown in FIG. 39, which is a partial cut-away side view of guard 3500 with a single-ended retractor 3510 inserted in the guard, guard 3500 also includes a contoured portion 3512 that at least partially follows the contour of the end of the retractor.

Each of the guards discussed above can be made from any desired material. Under most embodiments, the guards are formed from a plastic material having a durometer hardness of 40-80 shore measured on a D-type durometer or an A-type durometer. For some applications, the present inventors have found plastics with a durometer of 70 shore work well. In general, sharper instruments will require harder plastics in order to prevent the instruments from puncturing the guard. In general, the guards are formed of a material that can withstand sterilization so that the instrument is protected from shipping during and after sterilization.

The guards may be formed through any number of known processes including injection molding and dipping. The best process for any one guard will typically be determined based on the shape of the guard. After dipping or injection molding, holes may be cut into the guard to allow for better fluid flow through the guard or holes may be formed during the molding of the guard.

Although specific shapes for the guards have been discussed above, the invention is not limited to those shapes. Guards that provide a stable position for the guard while maintaining an opening defining portion above a surface are considered within the scope of the invention. In addition, guards that include a contoured portion that follows a contour of an instrument are considered within the scope of the present invention.

In addition, although the present invention has been described with reference to surgical retractors, the guards of the present invention are not limited to retractor guards.

Although the present invention has been described with reference to particular embodiments, workers skilled in the art will recognize that changes may be made in form and detail without departing from the spirit and scope of the invention.

What is claimed is:

1. A guard for a surgical instrument, the guard comprising:
an opening defining portion that defines an opening capable of receiving a portion of the surgical instrument; and
a base portion having a shape that maintains the guard in a stable position when the base portion is placed on a surface, the stable position such that the entirety of the opening defining portion is separated from the surface.

2. The guard of claim 1 wherein the base portion comprises a planar surface.

3. The guard of claim 1 wherein the base portion comprises a plurality of planar surfaces that each provide a separate stable position.

4. The guard of claim 1 wherein the base portion comprises a concave surface.

5. The guard of claim 1 further comprising a top portion extending from the opening defining portion such that a space is defined between the top portion and the base portion.

6. The guard of claim 5 wherein the top portion is parallel to the bottom portion.

7. The guard of claim 5 wherein the top portion extends toward the base portion as it extends away from the opening defining portion.

8. The guard of claim 1 wherein the base portion further comprises a second opening defining portion that defines a second opening in the guard.

9. A guard for a medical instrument, the guard comprising:
a bottom section comprising at least one substantially planar surface;
at least one side section extending from the bottom section; and
a top section extending from the side section and at least partially defining an opening that extends between two portions of the top section.

10. The guard of claim 9 wherein the at least one side section comprises a set of side walls extending from the perimeter of the bottom section.

11. The guard of claim 9 wherein the top section is parallel to the bottom section.

12. The guard of claim 9 wherein the bottom section defines a second opening.

13. The guard of claim 9 wherein the guard is formed as a single molded piece.

14. The guard of claim 9 wherein the opening is defined in part by a side section.

15. A guard for an end of a medical instrument, the end having a contoured shape, the guard comprising:
an opening that is large enough to permit insertion of the end of the medical instrument; and
a contoured portion having a contoured shape that at least partially follows the contour of the end of the medical instrument when the end is inserted in the guard.

16. The guard of claim 15 wherein the guard can withstand sterilization.

17. The guard of claim 15 wherein the guard further comprises a second opening.

18. The guard of claim 15 wherein the guard comprises a second contoured portion that at least partially follows a contour of a second end of the medical instrument when the second end is inserted in the guard.

19. The guard of claim 15 wherein the contoured portion is large enough to provide a space between the end of the medical instrument and the guard.
